(19) United States
(12) Patent Application Publication
(10) Pub. No.: US 2025/0161535 A1
(43) Pub. Date: May 22, 2025

(54) POLYMER SCAFFOLD FOR PROSTHESIS AND METHOD OF MANUFACTURING THE SAME

(71) Applicant: ARC KOREA CO., LTD., Seongnam-si, Gyeonggi-do (KR)

(72) Inventor: Jung-Wan SON, Seongnam-si, Gyeonggi-do (KR)

(21) Appl. No.: 18/841,348

(22) PCT Filed: Jan. 13, 2023
(86) PCT No.: PCT/KR2023/000617

§ 371 Date: Aug. 23, 2024

(30) Foreign Application Priority Data
Mar. 29, 2022 (KR) 10-2022-0038635

(51) Int. Cl. A61L 27/44; A61L 27/18; A61L 27/24; A61L 27/26; A61L 27/54; A61L 27/56; A61L 27/58

(52) U.S. Cl. CPC A61L 27/44; A61L 27/18; A61L 27/24; A61L 27/26; A61L 27/54; A61L 27/56; A61L 27/58; A61L 2430/00

(57) ABSTRACT

The present invention is to provide a polymer scaffold for a prosthesis includes two outer sheets formed into a certain size and shape and made of a biodegradable synthetic polymer material, and an inner sheet disposed between the two outer sheets and made of a biodegradable natural polymer material, wherein the two outer sheets are formed in the form of a single sealed pouch by joining the edges of the two outer sheets together, and the outer sheets are formed by cutting a portion of a synthetic polymer matrix made of the biodegradable synthetic polymer material and having a larger area and thickness than the outer sheets, and the inner sheet is formed by cutting a portion of a natural polymer matrix made of the biodegradable natural polymer material and having a larger area and thickness than the inner sheet.

### TECHNICAL FIELD

The present invention relates to a polymer scaffold for a prosthesis that is made of a biodegradable polymer material for stabilization and regeneration of an affected area and is implanted into the affected area to support the affected area, and a method of manufacturing the same.

### BACKGROUND ART

In the past, surgery was typically completed by wrapping the ligaments and tissues with sutures even when a space was created due to tissue damage or disconnection at the surgical site, such as the inside of muscle, joints, or the like.

For example, in the case of the rotator cuff repair shown in FIGS. 1 and 2, surgery is not performed while directly observing the inside of the joint, but is performed by incising a part of the joint and inserting an arthroscope into the joint while looking at the monitor screen.

As a result, the distance between the end of the surgical instrument and the hand is long, and the surgical instrument must be controlled indirectly through a monitor. Therefore, even when the surgeon is extremely skilled, a long surgery lasting more than an hour must be performed under general anesthesia.

Also, in order for the regenerative tissue at the surgical site to recover and the sutured state to be established after surgery, consistent rehabilitation movements must be performed within a set period of time.

However, when the muscles have not recovered because there is an empty space at the surgical site, the rehabilitation movements cause considerable pain, and thus a rehabilitation process cannot help to be a series of tremendous pain.

To solve this problem, as shown in U.S. Pat. No. 9,770,337 B2, a technology has been developed in the art to protect a surgical site by inserting a tube-shaped instrument into a surgical site to fill a liquid into the tube through a special nozzle during the surgical procedure and to ease the rehabilitation process by minimizing the movement of the surgical site during the rehabilitation process to significantly reduce pain.

However, in the related art, the leakage of the liquid filled in the tube must be prevented, so the nozzle used to inject the liquid into the tube must be installed with precision instruments having a special structure at the ends thereof so that no nozzle insertion holes are left in the tube after the nozzle is removed from the tube.

Also, since the tube must be prevented from melting inside the body in order to prevent the liquid leakage, a separate surgery to remove the tube is required later, which delays the patient's recovery and significantly increases costs.

In addition, the related art discloses that the ligament tissue repair function may be performed by attaching a biodegradable polymer (e.g., collagen, etc.) to the ligament during a ligament restoration procedure, but has problems in that the strength of the biodegradable hydrogel support is weak when used alone, and it is difficult to fix collagen and the like on the target body tissue surface, and also has a problem in that the collagen and the like easily become loose in the body after the procedure.

Therefore, there is a need for a technology that can reduce costs and eliminate stress by eliminating the need for subsequent removal surgery while maintaining the effect of stabilizing the surgical site and reducing pain, as in the related art.

In particular, although the drug delivery speed needs to be adjusted because the speed of recovery or tissue regeneration varies depending on the type of affected area or the age of a patient, it is still difficult to find a technology for biodegradable drug delivery and tissue regeneration scaffolds with a means capable of controlling the drug delivery speed.

Further, although the type and size of the affected area may both differ depending on the type and severity of the injury, and the recovery speed and tissue regeneration speed may both differ depending on the age, body type, and physique of a patient even when patients have the same type of injury having similar severity, the technology for implantable prostheses capable of being provided in a customized manner to patients is still inadequate.

### RELATED-ART DOCUMENT

U.S. Pat. No. 9,770,337 (Registration date: Sep. 26, 2017)

### DISCLOSURE

### Technical Problem

Therefore, the present invention is directed to providing a polymer scaffold for a prosthesis, which is an implantable support that may be provided in a customized manner to patients because the type and size of the affected area are both different depending on the type and severity of the injury, and the recovery speed and tissue regeneration speed may both be different depending on the age, body type, and physique of a patient even when patients have the same type of injury having similar severity, and a method of manufacturing the same.

### Technical Solution

To solve the above problems, a polymer scaffold for a prosthesis according to the present invention includes two outer sheets formed into a certain size and shape and made of a biodegradable synthetic polymer material, and an inner sheet disposed between the two outer sheets and made of a biodegradable natural polymer material, wherein the two outer sheets are formed in the form of a single sealed pouch by joining the edges of the two outer sheets together, and the outer sheets are formed by cutting a portion of a synthetic polymer matrix made of the biodegradable synthetic polymer material and having a larger area and thickness than the outer sheets, and the inner sheet is formed by cutting a portion of a natural polymer matrix made of the biodegradable natural polymer material and having a larger area and thickness than the inner sheet.

Here, the edges of the two outer sheets may be preferably joined by heat pressing so that the edges can be formed into a heat-bonded band having a certain width.

Also, the synthetic polymer matrix may be preferably formed into a fine network structure through amorphous stacking of synthetic polymer fibers that are injected by applying a high voltage to a piston nozzle, and a plurality of synthetic polymer matrices having different densities and porosities may be provided by differently forming the thickness of the fibers and the stacking distance between the fibers, depending on the viscosity of a synthetic polymer solution filled in the piston nozzle and the magnitude of the high voltage, so that the outer sheets can be provided with a plurality of sheets having different densities and porosities.

In addition, the material of the synthetic polymer matrix may include a component composed of any one or a combination of two or more selected from poly(L-lactic acid) (PLA), poly(glycolic acid) (PLGA), poly(ε-caprolactone) (PCL), and poly(L-lactide-co-ε-carprolactone (PLCL).

Additionally, the material of the synthetic polymer matrix may preferably include a component composed of any one or a combination of two or more selected from collagen type 1, sodium hyaluronate (HA), and chondroitin sulfate.

Further, the inner sheet may preferably contain a therapeutic drug regenerating an affected tissue.

Meanwhile, a method of manufacturing a polymer scaffold according to the present invention includes mixing a biodegradable natural polymer fuel to prepare a natural polymer solution and then drying the natural polymer solution to manufacture a natural polymer matrix; mixing a biodegradable synthetic polymer fuel to prepare a synthetic polymer solution and then manufacturing a synthetic polymer matrix using the synthetic polymer solution; manufacturing an inner sheet by cutting the natural polymer matrix to an area and thickness corresponding to an affected area in which the inner sheet will be implanted; manufacturing two outer sheets by cutting the synthetic polymer matrix twice to a certain area and thickness so as to have a larger area than the inner sheet; disposing the inner sheet between the two outer sheets; and joining the two outer sheets along the edges thereof.

Here, the manufacturing of the synthetic polymer matrix may preferably include dispersing the synthetic polymer solution by spraying the synthetic polymer solution through a piston nozzle, wherein the synthetic polymer sprayed from the nozzle may be formed in the form of fine fiber strands while weakening the surface tension of the synthetic polymer by disposing a high-voltage electrode at the nozzle and applying a high voltage to the nozzle, and the fiber form may be produced by sequentially amorphously stacking the fiber strands on a predetermined collector plate having a certain area.

In this case, the manufacturing of the synthetic polymer matrix may preferably include adjusting the porosity and density of the completed synthetic polymer matrix by increasing or decreasing the viscosity of the synthetic polymer solution to reduce or increase the thickness of the fiber strands, increasing or decreasing the magnitude of the high voltage to reduce or increase the thickness of the fiber strands, and decreasing or increasing the inflow speed of the synthetic polymer solution introduced into the piston nozzle to reduce or increase the thickness of the fiber strands.

Also, the manufacturing of the synthetic polymer matrix may preferably include further decreasing the diameter of the fiber strands spun from the piston nozzle by adding a salt to the synthetic polymer solution to increase the surface charge density of the synthetic polymer sprayed from the piston nozzle.

Meanwhile, the joining of the two outer sheets along the edges thereof may preferably include bringing the edges of the two outer sheets into close contact with each other, and joining the outer sheets by applying heat along a region where the edges of the outer sheets come into close contact, wherein even when the outer sheets and the inner sheet are manufactured to have an area corresponding to the size and shape of a specific affected area, the outer sheets and the inner sheet may be manufactured in the form of a sealed pouch so that the outer sheets and the inner sheet can be quickly implanted into the affected area.

### Advantageous Effects

The polymer scaffold for a prosthesis and the method of manufacturing the same according to the present invention have an effect of being able to be provided in a customized manner to patients because the type and size of the affected area may both be different depending on the type and severity of the injury, and the recovery speed and tissue regeneration speed may both be different depending on the age, body type, and physique of a patient even when patients have the same type of injury having similar severity.

### DESCRIPTION OF DRAWINGS

FIGS. 1 and 2 are diagrams showing conventional rotator cuff repair.

FIG. 3 is a perspective view of a polymer scaffold for a prosthesis according to the present invention.

FIG. 4 is a cross-sectional side view of FIG. 3.

FIG. 5 is a plan view of FIG. 3.

FIG. 6 is a conceptual diagram showing electrospinning in a method of manufacturing a polymer scaffold for a prosthesis according to the present invention.

FIG. 7 is a conceptual diagram showing some principles for electrospinning of FIG. 6.

FIG. 8 is a conceptual diagram showing the latter process of the method of manufacturing a polymer scaffold for a prosthesis according to the present invention.

FIG. 9 is a flow chart showing the method of manufacturing a polymer scaffold for a prosthesis according to the present invention.

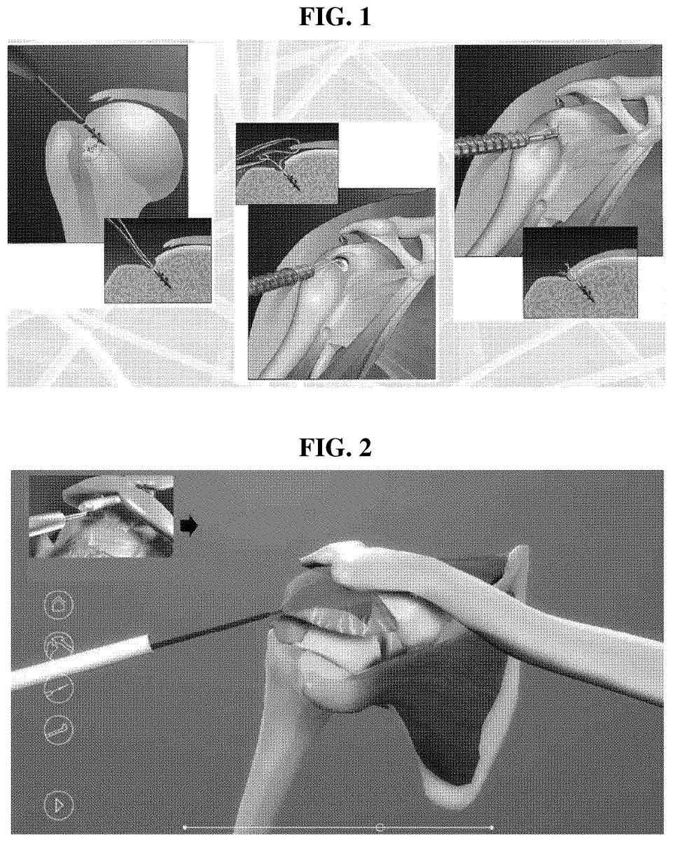

### MODE FOR INVENTION

Specific structural or functional descriptions presented in embodiments of the present invention are merely illustrative for the purpose of describing the embodiments according to the concept of the present invention, and the embodiments according to the concept of the present invention may be implemented in various forms. Also, it should be understood that the present invention is not intended to be limited to the embodiments described in this specification, and includes all modifications, equivalents, and substitutions which fall within the spirit and technical scope of the present invention.

Hereinafter, the present invention will be described in detail with reference to the accompanying drawings.

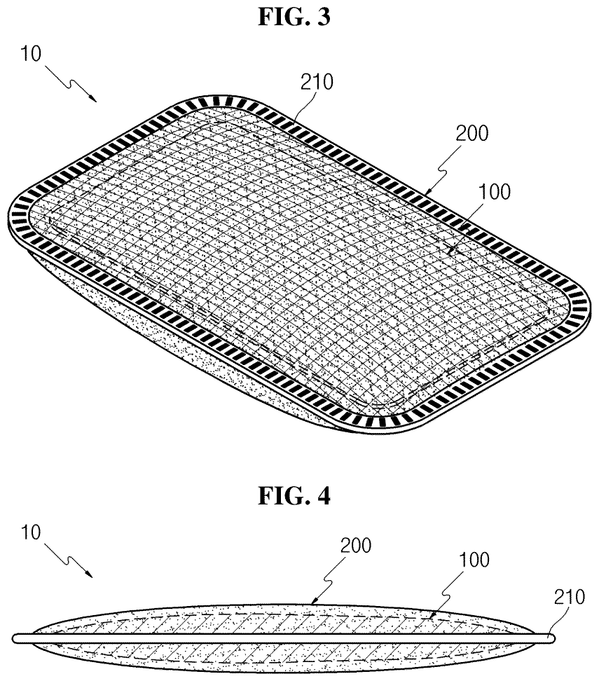

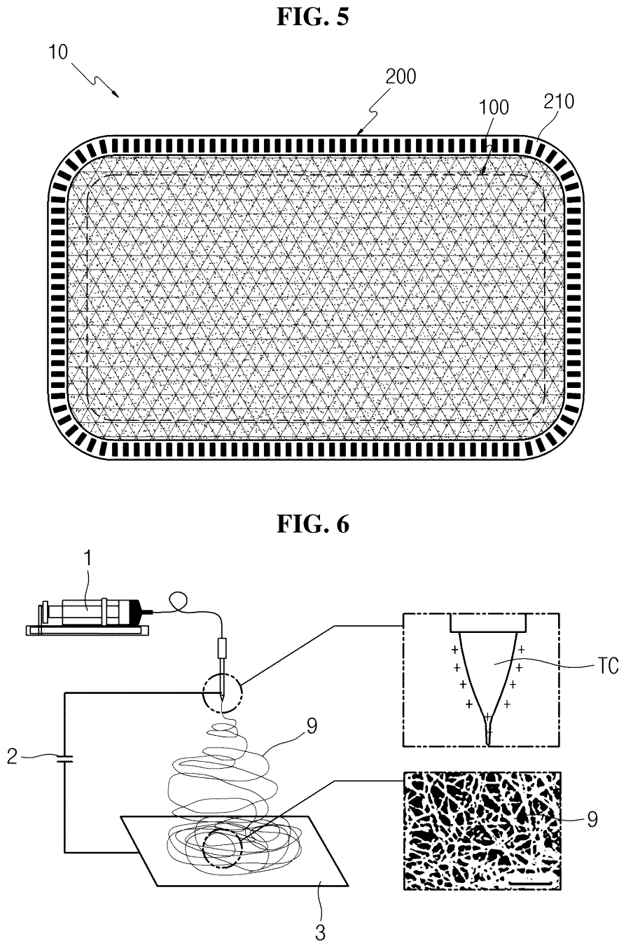

A polymer scaffold 10 for a prosthesis according to the present invention is composed of two outer sheets 200 formed into a certain size and shape and made of a biodegradable synthetic polymer material, and an inner sheet 100 disposed between the two outer sheets 200 and made of a biodegradable natural polymer material, as shown in FIGS. 3 to 5.

Here, the two outer sheets 200 are formed in the form of a single sealed pouch by joining the edges of the two outer sheets together, wherein the outer sheets 200 may be formed by cutting a portion of a synthetic polymer matrix 8 made of the biodegradable synthetic polymer material and having a larger area and thickness than the outer sheets 200, and the inner sheet 100 may be formed by cutting a portion of a natural polymer matrix 7 made of the biodegradable natural polymer material and having a larger area and thickness than the inner sheet 100.

That is, the outer sheets 200 made of the biodegradable synthetic polymer and the inner sheet 100 made of the biodegradable natural polymer are first manufactured in the form of a relatively large matrix having a certain size and thickness. Then, when there is a patient requiring immediate implantation, these sheets are cut to an actually required size and shape corresponding to the affected area of the patient depending on the size of the space where the prosthesis should be implanted in the affected area of the patient, the severity of the injury, the recovery period according to the age of the patient, and the like. Accordingly, the implantable prosthesis may be prepared in a customized manner for the first time.

In this case, the two outer sheets 200 are formed in the form of a single sealed pouch by joining the edges of the two outer sheets together so that the inner sheet 100 can be prevented from rapidly dissolving when a body fluid seeps in and can be decomposed at a constant rate while passing through the microstructure constituting the outer sheets 200.

In particular, as the inner sheet 100 is dissolved in body fluid, the inner sheet 100 may escape through the outer sheets 200 together with the therapeutic drug contained in the inner sheet 100 while passing through the microstructure constituting the outer sheets 200. Here, since the speed at which the therapeutic drug also escapes to the outside may be controlled according to the microstructure characteristics of the outer sheets 200, it is possible to supply the therapeutic drug to the affected area at a speed suited to the characteristics of the patient and the characteristics of the affected area.

Also, the synthetic polymer matrix 8 corresponding to the raw material of the outer sheets 200 may be formed in the form of a network structure in which strands of nanofibers 9 having a fine tissue structure are amorphously stacked. In this case, the fine strands of nanofibers 9 may be extracted in an electrospun form, which will be described later.

In this way, when the outer sheets 200 are extracted in an electrospun form so that the outer sheets 200 have a structure in which the fine strands of nanofibers 9 are amorphously stacked, the polymer scaffold 10 for a prosthesis according to the present invention may be implanted into the affected area. At this time, first, since the speed at which the inner sheet 100 embedded in the outer sheets 200 and a drug contained in the inner sheet 100 are discharged through the outer sheets 200 may be controlled, it is possible to manufacture a customized polymer scaffold 10 for a prosthesis to correspond to the tissue regeneration speed according to the nature of the affected area, the physical age-specific characteristics of a patient, the severity of the affected area, and the like. Second, since the speed at which the outer sheets 200 self-dissolve after the material in the outer sheets 200 is discharged may be controlled, the polymer scaffold 10 for a prosthesis according to the present invention may serve to support the affected area by shrinking at an appropriate speed without applying unnecessary pressure to the affected area depending on the degree of healing of the affected area.

In this case, the edges of the two outer sheets 200 may be joined by heat pressing so that the edges can be formed into a heat-bonded band 210 having a certain width. That is, although the outer sheets 200 may be cut and formed on the spot to any size and heat is applied to join the two outer sheets 200 along the edges thereof at a certain width from the edges, the edges themself may be immediately formed in the form of a sealed pouch while being formed into a heat-bonded band 210.

Therefore, the formation of the heat-bonded band 210 means that the polymer scaffold 10 for a prosthesis according to the present invention may be manufactured on the spot into a customized prosthesis that fits an actual specific patient. In this case, this effect is achieved by providing the heat-bonded band 210 without requiring any other separate configurations for sealing.

Also, when the outer sheets 200 are made of a material in which fine nanofibers 9 are stacked as described above, the synthetic polymer matrix 8 may be formed into a fine network structure through amorphous stacking of the synthetic polymer nanofibers 9 that are injected by applying a high voltage to a piston nozzle 1 so that the density and porosity of the outer sheets 200 can be controlled.

In this case, a plurality of synthetic polymer matrices 8 having different densities and porosities may be provided by differently forming the thickness of the nanofibers 9 and the stacking distance between the nanofibers 9, depending on the viscosity of a synthetic polymer solution filled in the piston nozzle 1 and the magnitude of the high voltage, so that the outer sheets 200 can be provided with a plurality of outer sheets having different densities and porosities.

For reference, the material of the synthetic polymer matrix 8 used herein may include a component composed of any one or a combination of two or more selected from poly(L-lactic acid) (PLA), poly(glycolic acid) (PLGA), poly(ε-caprolactone) (PCL), and poly(L-lactide-co-ε-carprolactone (PLCL).

Also, the material of the natural polymer matrix 7 may include a component composed of any one or a combination of two or more selected from collagen type 1, sodium hyaluronate (HA), and chondroitin sulfate.

Hereinafter, a method of manufacturing a polymer scaffold for a prosthesis according to the present invention will be described.

The method of manufacturing a polymer scaffold for a prosthesis according to the present invention includes mixing a biodegradable natural polymer fuel to prepare a natural polymer solution and then drying the natural polymer solution to manufacture a natural polymer matrix 7, mixing a biodegradable synthetic polymer fuel to prepare a synthetic polymer solution and then manufacturing a synthetic polymer matrix 8 using the synthetic polymer solution, manufacturing an inner sheet 100 by cutting the natural polymer matrix 7 to an area and thickness corresponding to an affected area in which the inner sheet will be implanted, manufacturing two outer sheets 200 by cutting the synthetic polymer matrix 8 twice to a certain area and thickness so as to have a larger area than the inner sheet 100, disposing the inner sheet 100 between the two outer sheets 200, and joining the two outer sheets 200 along the edges thereof.

At this time, as described above, the outer sheets 200 are manufactured in the form of a synthetic polymer matrix 8 and then cut therefrom, thereby enabling the manufacture of the outer sheets 200 to a customized size. In this case, the synthetic polymer matrix may be manufactured using an electrospinning method to control the porosity and density.

Therefore, the manufacturing of the synthetic polymer matrix 8 may include dispersing the synthetic polymer solution by spraying the synthetic polymer solution through a piston nozzle 1, as shown in FIG. 6, wherein the synthetic polymer matrix 8 may be manufactured using an electrospinning method by which the synthetic polymer sprayed from the nozzle 1 is formed in the form of fine nanofiber 9 strands while weakening the surface tension of the synthetic polymer by disposing a high-voltage electrode 2 at the nozzle 1 and applying a high voltage to the nozzle 1, and the fiber form is produced by sequentially amorphously stacking the fiber strands on a predetermined collector plate 3 having a certain area.

In this case, the manufacturing of the synthetic polymer matrix 8 may include adjusting the porosity and density of the completed synthetic polymer matrix 8 by increasing or decreasing the viscosity of the synthetic polymer solution to reduce or increase the thickness of the fiber strands, increasing or decreasing the magnitude of the high voltage to reduce or increase the thickness of the fiber strands, and decreasing or increasing the inflow speed of the synthetic polymer solution introduced into the piston nozzle 1 to reduce or increase the thickness of the fiber strands.

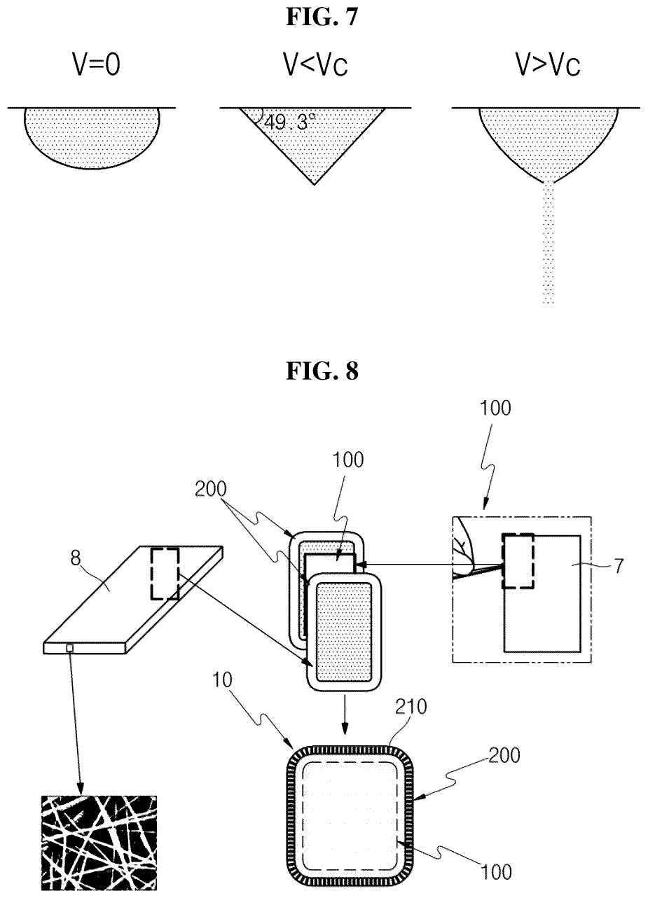

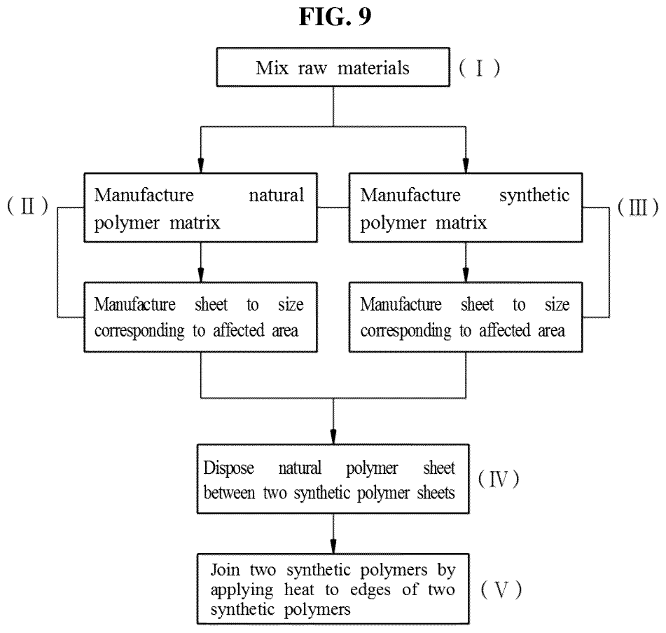

In this case, as shown in FIG. 6, when a charge is injected into the discharged polymer solution to charge the polymer solution and an opposite electrode is connected to the collector plate 3, the polymer solution forms a hemisphere due to the surface tension of the polymer discharged from the tip of the nozzle. At this time, as shown in FIG. 7, a liquid-phase polymer solution droplet is elongated into a funnel shape similar to a cone due to the mutual electrostatic repulsion between the surface charges caused by the high voltage and the Coulomb force applied to the external electric field. Here, as the charge continues to accumulate in the polymer solution, the spinning-elongation takes place by a Taylor cone-shaped jet due to the mutual repulsion of the same charge, as shown on the right side of FIG. 6. In this case, the fibers are gathered in the direction of the collector plate (3) that is charged with the opposite charge or grounded.

In this case, the diameter of nanofibers manufactured by electrospinning is greatly affected by the spinning conditions.

The first factor of the spinning conditions is the voltage. High voltage may cause a larger amount of the polymer solution to be introduced into the jet, which may increase the diameter of the nanofibers. Also, in the case of natural polymers such as silk, the diameter of the nanofibers may decrease as the voltage increases. In particular, the high surface charge density induces high mobility of ions, which leads to a large electrostatic repulsion, thereby greatly increasing elongation and further decreasing the diameter of the nanofibers.

The second factor is the viscosity of the polymer solution. In electrospinning, in the case of a solution with low viscosity, the polymer accumulates in the form of droplets due to surface tension. As the concentration gradually increases, the polymer flies through the air without any collapse, and the spindle-shaped droplets are connected to each other by fine threads to form stable continuous fibers. Also, as the viscosity of the solution increases, the degree of entanglement of the polymer chains in a solvent increases, which prevents the collapse of the jet (cone jet, liquid jet, or initial jet). As a result, the jet takes the form of elongated fibers.

When the speed at which the polymer solution is introduced into the piston nozzle is controlled to make the inflow speed faster, the size of the droplets formed at the tip of the nozzle increases, and even when the droplets reach the collector plate 3 after spinning, the solvent is not completely evaporated. Therefore, the finally obtained layer of nanofibers 9 may include a structure in which bead-shaped fibers or multiple strands of fibers intersect each other.

Therefore, the smaller the inflow speed of the polymer solution, the smaller the diameter of the obtained nanofibers 9, and the larger the inflow speed of the polymer solution, the larger the diameter of the obtained nanofibers 9.

Also, the manufacturing of the synthetic polymer matrix 8 may include further decreasing the diameter of the nanofiber 9 strands spun from the piston nozzle 1 by adding a small amount of salt to the synthetic polymer solution to increase the surface charge density of the synthetic polymer sprayed from the piston nozzle.

This is because the added salt induces a higher charge density to be formed on the surface of the polymer solution to be discharged. This is because the mobility of the polymer solution increases further and a larger electrostatic repulsion force is generated, resulting in larger elongation.

Therefore, according to the method of manufacturing a polymer scaffold 10 for a prosthesis according to the present invention, the porosity and density of the outer sheets may be controlled so that an optimal prosthesis can be provided to an affected area of an actual specific patient, and a plurality of synthetic polymer matrices and natural polymer matrices having different porosities and densities are provided so that a prosthesis whose dissolution rate can be controlled so as to be optimized for the patient's treatment period and tissue regeneration period can be provided.

The present invention described above is not limited to the above-described embodiments and the accompanying drawings, and it will be apparent to one of ordinary skill in the art to which the present invention pertains that various substitutions, modifications and changes are possible without departing from the technical scope of the present invention.

[Brief Description of Main Parts]

| | |
|---|---|
| TC: Taylor cone shape | 1: piston nozzle |
| 2: high-voltage electrode | 3: collector plate |
| 7: natural polymer matrix | 8: synthetic polymer matrix |
| 9: nanofibers | |

10: polymer scaffold for a prosthesis according to the present invention

| | |
|---|---|
| 100: inner sheet | 200: outer sheet |
| 210: heat-bonded band | |

1. A polymer scaffold for a prosthesis comprising:
two outer sheets formed into a certain size and shape and made of a biodegradable synthetic polymer material; and
an inner sheet disposed between the two outer sheets and made of a biodegradable natural polymer material,
wherein the two outer sheets are formed in the form of a single sealed pouch by joining the edges of the two outer sheets together,
the outer sheets are formed by cutting a portion of a synthetic polymer matrix made of the biodegradable synthetic polymer material and having a larger area and thickness than the outer sheets,
the inner sheet is formed by cutting a portion of a natural polymer matrix made of the biodegradable natural polymer material and having a larger area and thickness than the inner sheet, and
the outer sheets have a network-type structure made through amorphous stacking of fine fibers.

2. The polymer scaffold of claim 1, wherein the edges of the two outer sheets are joined by heat pressing so that the edges are formed into a heat-bonded band having a certain width.

3. The polymer scaffold of claim 1, wherein the synthetic polymer matrix is formed into a fine network structure through amorphous stacking of synthetic polymer fibers that are injected by applying a high voltage to a piston nozzle, and
a plurality of synthetic polymer matrices having different densities and porosities are provided by differently forming the thickness of the fibers and the stacking distance between the fibers, depending on the viscosity of a synthetic polymer solution filled in the piston nozzle and the magnitude of the high voltage, so that the outer sheets are provided with a plurality of sheets having different densities and porosities.

4. The polymer scaffold of claim 1, wherein the material of the synthetic polymer matrix includes a component composed of any one or a combination of two or more selected from poly(L-lactic acid) (PLA), poly(glycolic acid) (PLGA), poly(ε-caprolactone) (PCL), and poly(L-lactide-co-ε-carprolactone (PLCL).

5. The polymer scaffold of claim 1, wherein the material of the natural polymer matrix includes a component composed of any one or a combination of two or more selected from collagen type 1, sodium hyaluronate (HA), and chondroitin sulfate.

6. The polymer scaffold of claim 1, wherein the inner sheet contains a therapeutic agent for regenerating an affected tissue.

7. A method of manufacturing a polymer scaffold for a prosthesis, comprising:
mixing a biodegradable natural polymer fuel to prepare a natural polymer solution and then drying the natural polymer solution to manufacture a natural polymer matrix;
mixing a biodegradable synthetic polymer fuel to prepare a synthetic polymer solution and then manufacturing a synthetic polymer matrix using the synthetic polymer solution;
manufacturing an inner sheet by cutting the natural polymer matrix to an area and thickness corresponding to an affected area in which the inner sheet will be implanted;
manufacturing two outer sheets by cutting the synthetic polymer matrix twice to a certain area and thickness so as to have a larger area than the inner sheet;
disposing the inner sheet between the two outer sheets; and
joining the two outer sheets along the edges thereof.

8. The method of claim 7, wherein the manufacturing of the synthetic polymer matrix includes dispersing the synthetic polymer solution by spraying the synthetic polymer solution through a piston nozzle, wherein the synthetic polymer sprayed from the nozzle is formed in the form of fine fiber strands while weakening the surface tension of the synthetic polymer by disposing a high-voltage electrode at the nozzle and applying a high voltage to the nozzle, and the fiber form is produced by sequentially amorphously stacking the fiber strands on a predetermined collector plate having a certain area.

9. The method of claim 8, wherein the manufacturing of the synthetic polymer matrix includes adjusting the porosity and density of the completed synthetic polymer matrix by increasing or decreasing the viscosity of the synthetic polymer solution to reduce or increase the thickness of the fiber strands, increasing or decreasing the magnitude of the high voltage to reduce or increase the thickness of the fiber strands, and decreasing or increasing the inflow speed of the synthetic polymer solution introduced into the piston nozzle to reduce or increase the thickness of the fiber strands.

10. The method of claim 8, wherein the manufacturing of the synthetic polymer matrix includes further decreasing the diameter of the fiber strands spun from the piston nozzle by adding a salt to the synthetic polymer solution to increase the surface charge density of the synthetic polymer sprayed from the piston nozzle.

11. The method of claim 7, wherein the joining of the two outer sheets along the edges thereof includes:
bringing the edges of the two outer sheets into close contact with each other, and
joining the outer sheets by applying heat along a region where the edges of the outer sheets come into close contact,
wherein even when the outer sheets and the inner sheet are manufactured to have an area corresponding to the size and shape of a specific affected area, the outer sheets and the inner sheet are able to be manufactured in the form of a sealed pouch so that the outer sheets and the inner sheet can be quickly implanted into the affected area.
